SEQUENCE LISTING (15 sequences)

SEQ ID NO: 1 - protein, 125 aa, Artificial Sequence
  LC3B
MPSEKTFKQRRTFEQRVEDVRLIREQHPTKIPVIIERYKGEKQLPVLDKTKFLVPDHVNMSELIKIIRRRLQLNANQAFFLLVNGHSMVSVSTPISEVYESEKDEDGFLYMVYASQETFGMKLSV

SEQ ID NO: 2 - protein, 356 aa, Artificial Sequence
  GST-LC3B
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLEVLFQGPLGSMPSEKTFKQRRTFEQRVEDVRLIREQHPTKIPVIIERYKGEKQLPVLDKTKFLVPDHVNMSELIKIIRRRLQLNANQAFFLLVNGHSMVSVSTPISEVYESEKDEDGFLYMVYASQETFGMKLSV

SEQ ID NO: 3 - protein, 130 aa, Artificial Sequence
  LC3B(1-125)
GPLGSMPSEKTFKQRRTFEQRVEDVRLIREQHPTKIPVIIERYKGEKQLPVLDKTKFLVPDHVNMSELIKIIRRRLQLNANQAFFLLVNGHSMVSVSTPISEVYESEKDEDGFLYMVYASQETFGMKLSV

SEQ ID NO: 4 - protein, 123 aa, Artificial Sequence
  LC3B(2-119)
GSPEFPSEKTFKQRRTFEQRVEDVRLIREQHPTKIPVIIERYKGEKQLPVLDKTKFLVPDHVNMSELIKIIRRRLQLNANQAFFLLVNGHSMVSVSTPISEVYESEKDEDGFLYMVYASQETF

SEQ ID NO: 5 - protein, 15 aa, Artificial Sequence
  N-terminal FITC-labeled peptide
GGDDDWTHLSSKEVD

SEQ ID NO: 6 - protein, 15 aa, Artificial Sequence
  LBP2
GGDDDWTHLSSKEVD

SEQ ID NO: 7 - protein, 130 aa, Artificial Sequence
  K8A
GPLGSMPSEKTFAQRRTFEQRVEDVRLIREQHPTKIPVIIERYKGEKQLPVLDKTKFLVPDHVNMSELIKIIRRRLQLNANQAFFLLVNGHSMVSVSTPISEVYESEKDEDGFLYMVYASQETFGMKLSV

SEQ ID NO: 8 - protein, 130 aa, Artificial Sequence
  K30A
GPLGSMPSEKTFKQRRTFEQRVEDVRLIREQHPTAIPVIIERYKGEKQLPVLDKTKFLVPDHVNMSELIKIIRRRLQLNANQAFFLLVNGHSMVSVSTPISEVYESEKDEDGFLYMVYASQETFGMKLSV

SEQ ID NO: 9 - protein, 130 aa, Artificial Sequence
  K39A
GPLGSMPSEKTFKQRRTFEQRVEDVRLIREQHPTKIPVIIERYAGEKQLPVLDKTKFLVPDHVNMSELIKIIRRRLQLNANQAFFLLVNGHSMVSVSTPISEVYESEKDEDGFLYMVYASQETFGMKLSV

SEQ ID NO: 10 - protein, 130 aa, Artificial Sequence
  K42A
GPLGSMPSEKTFKQRRTFEQRVEDVRLIREQHPTKIPVIIERYKGEAQLPVLDKTKFLVPDHVNMSELIKIIRRRLQLNANQAFFLLVNGHSMVSVSTPISEVYESEKDEDGFLYMVYASQETFGMKLSV

SEQ ID NO: 11 - protein, 130 aa, Artificial Sequence
  K49A
GPLGSMPSEKTFKQRRTFEQRVEDVRLIREQHPTKIPVIIERYKGEKQLPVLDATKFLVPDHVNMSELIKIIRRRLQLNANQAFFLLVNGHSMVSVSTPISEVYESEKDEDGFLYMVYASQETFGMKLSV

SEQ ID NO: 12 - protein, 130 aa, Artificial Sequence
  K51A
GPLGSMPSEKTFKQRRTFEQRVEDVRLIREQHPTKIPVIIERYKGEKQLPVLDKTAFLVPDHVNMSELIKIIRRRLQLNANQAFFLLVNGHSMVSVSTPISEVYESEKDEDGFLYMVYASQETFGMKLSV

SEQ ID NO: 13 - protein, 130 aa, Artificial Sequence
  K65A
GPLGSMPSEKTFKQRRTFEQRVEDVRLIREQHPTKIPVIIERYKGEKQLPVLDKTKFLVPDHVNMSELIAIIRRRLQLNANQAFFLLVNGHSMVSVSTPISEVYESEKDEDGFLYMVYASQETFGMKLSV

SEQ ID NO: 14 - protein, 130 aa, Artificial Sequence
  K103A
GPLGSMPSEKTFKQRRTFEQRVEDVRLIREQHPTKIPVIIERYKGEKQLPVLDKTKFLVPDHVNMSELIKIIRRRLQLNANQAFFLLVNGHSMVSVSTPISEVYESEADEDGFLYMVYASQETFGMKLSV

SEQ ID NO: 15 - protein, 130 aa, Artificial Sequence
  K122A
GPLGSMPSEKTFKQRRTFEQRVEDVRLIREQHPTKIPVIIERYKGEKQLPVLDKTKFLVPDHVNMSELIKIIRRRLQLNANQAFFLLVNGHSMVSVSTPISEVYESEKDEDGFLYMVYASQETFGMALSV

(19) United States
(12) Patent Application Publication
(10) Pub. No.: US 2020/0069609 A1
(43) Pub. Date: Mar. 5, 2020

(54) METHOD FOR COVALENT BOND MODIFYING MAMMALIAN ATG8 HOMOLOGUE

(71) Applicant: WIGEN BIOMEDICINE TECHNOLOGY (SHANGHAI) CO., LTD., Shanghai (CN)

(72) Inventors: Cheng LUO, Shanghai (CN); Zhiyi YAO, Shanghai (CN); Yuli XIE, Shanghai (CN); Wei WAN, Shanghai (CN); Liyan YUE, Shanghai (CN); Yuanyuan ZHANG, Shanghai (CN); Hualiang JIANG, Shanghai (CN); Kaixian CHEN, Shanghai (CN)

(21) Appl. No.: 16/614,481

(22) PCT Filed: May 18, 2018
(86) PCT No.: PCT/CN2018/087449

§ 371 Date: Nov. 18, 2019

(30) Foreign Application Priority Data
May 22, 2017 (CN) 201710364918.8

(51) Int. Cl. A61K 31/122; C07K 1/107; C07K 14/47; C07K 19/00

(52) U.S. Cl. CPC A61K 31/122; C07K 19/00; C07K 14/47; C07K 1/107

(57) ABSTRACT

The present invention provides a method (I) for modifying a mammalian ATG8 homologue by a covalent bond, comprising: providing a compound SM-LG including a moiety SM- having a function of modulating a mammalian ATG8 homologue and a leaving moiety -LG; the compound SM-LG reacts with a mammalian ATG8 homologue to produce a covalent complex of the mammalian ATG8 homologue. The invention also provides a covalent complex of the mammalian ATG8 homologue obtained by the method and uses of the same.

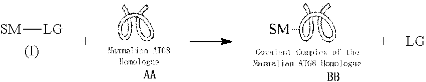

### FIELD OF THE INVENTION

The invention relates to a method for modulating a mammalian ATG8 homologue, particularly relating to a method for modifying a mammalian ATG8 homologue with a covalent bond, the covalent complex of the mammalian ATG8 homologue prepared by the same and its uses.

### BACKGROUND OF THE INVENTION

Autophagy is a cellular degradative pathway whereby dysfunctional proteins or organelles are transported to lysosome and then digested and degraded. It is a universal and conservative process amongst yeast, plants and mammals during the process of biological evolution.

Current studies demonstrate that autophagy not only plays an important part in maintaining physiological functions, such as providing nutrients, eliminating cells contents, antigen presentation, but also has key functions in diseases such as cancers, infectious diseases and neurodegenerative disorders.

In the developing process of tumors, the autophagy functions as a double edged-sword role: in the early stage of tumor development, the autophagy defects may increase genomic instabilities and promote carcinogenesis; in tumor rapidly growing and metastasis stages, autophagy can resist stress conditions to inhibit anoikis and maintain tumor cell survival. Although the relationship between autophagy and tumors varies at different stages of tumor development, the development of autophagy modulators will be of great value for advanced cancers and chemotherapy-resistant cancers.

Currently, there are about 30 clinical trials about autophagy modulation, for example, using hydroxychloroquine alone, chloroquine alone or using combined with other anti-tumor drugs to assess the therapeutic effects of autophagy inhibition mainly on refractory or relapsed solid tumors. Relevant results can be retrieved on the clinicaltrial.gov website. However, the side effects of antilysosomal agents and undetermined directions of chemical space optimization may severely limit further development of these types of autophagy inhibitors, because of a lack of definite molecular targets.

Small molecules modulators targeting autophagy are focused on mTOR or lysosome modulators at present. Small molecules modulators of autophagy related proteins, like the enzymes ATG4 and ULK1, are still at an early development stage. ATG8 and its mammalian homologous family proteins LC3, GABARAP and GATE-16 subfamilies are the most important autophagy related proteins. In human body, the LC3 family has LC3A, LC3B and LC3C; the GABARAP family has GABARAP and GABARAPL1; and the GATE-16 family has GABARAPL2. LC3B is undoubtedly the one has been studied most completely among the ATG8 mammalian homologous proteins. It is believed to be a marker of autophagy.

The method for developing covalently modified a mammalian ATG8 homologue is beneficial for studying the function of the protein for modulating themselves and their function mechanism in autophagy, and is good for developing modulators for regulating autophagy and medicants for treating autophagy-related diseases.

### SUMMARY OF THE INVENTION

The present invention provides a method for modulating a mammalian ATG8 homologue, comprising: providing a compound SM-LG including a moiety SM- having a function of modulating a mammalian ATG8 homologue and a leaving moiety -LG; the compound SM-LG reacts with a mammalian ATG8 homologue to produce a covalent complex of the mammalian ATG8 homologue.

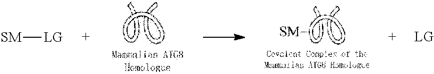

In a more specific embodiment of the invention, the reaction of the compound SM-LG with a mammalian ATG8 homologue is a substitution reaction.

In a more specific embodiment of the invention, the reaction of the compound SM-LG with a mammalian ATG8 homologue is a nucleophilic substitution reaction.

In a more specific embodiment of the invention, LG-H is a small molecule compound, preferably a water molecule; and SM- has a structure of α,β-unsaturated carbonyl.

The invention also provides a covalent complex of a mammalian ATG8 homologue having the following structure:

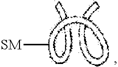

wherein, [symbol] is a mammalian ATG8 homologue, SM- is a moiety that has a function of modulating a mammalian ATG8 homologue; preferably, SM- has a structure of α,β-unsaturated carbonyl.

In the covalent complex of the mammalian ATG8 homologue, SM- is linked to the mammalian ATG8 homologue by a covalent bond.

In a more specific embodiment of the invention, the covalent complex of the mammalian ATG8 homologue has the following structure:

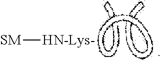

SM is linked to the ε-amino group of the first lysine at positions 46-55 in the mammalian ATG8 homologue by a covalent bond, wherein HN-Lys- represents the ε-amino group of the first lysine at positions 46-55 in a mammalian ATG8 homologue.

In a more specific embodiment of the invention, the mammalian ATG8 homologue is LC3B, preferably SM- is linked to the ε-amino group of the lysine at position 49 in LC3B by a covalent bond.

In a more specific embodiment of the invention, said SM has the structure as shown in the following general formula Ia:

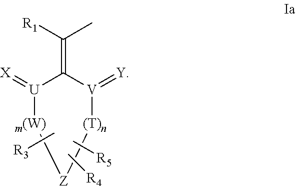

In the general formula Ia:

X and Y are each independently selected from the group consisting of O, S, NRₐ, NOH, and CH₂;

U and V are each independently selected from the group consisting of C, S, SO, and PORₐ;

W, Z, and T are each independently selected from the group consisting of O, S, SO, SO₂, N, NRₐ, CO, C, CRₐ, and CH₂;
Rₐ is H or C1-6 alkyl;
m is 0, 1, 2, or 3;
n is 0, 1, 2, or 3;

R₁ is selected from the group consisting of H, deuterium, unsubstituted C1-6 alkyl or C1-6 alkyl substituted by a substituent selected from hydroxyl and halogen, unsubstituted phenyl or phenyl substituted by a substituent selected from halogen, hydroxyl, C1-C6 alkyl and C1-C6 heteroalkyl;

R₃, R₄, and R₅ are each independently selected from the group consisting of H; hydroxyl; amino group; halogen; cyano; nitro; carboxyl; formyl; amide group; ester group; unsubstituted C1-6 alkyl or C1-6 alkyl substituted by a substituent selected from hydroxyl, halogen and C1-6 alkoxy; C1-6 heteroalkyl; C2-6 alkenyl; C2-6 alkynyl; substituted or unsubstituted —CONH₂—(C6-10 aryl); substituted or unsubstituted —CH═CH—(C6-10 aryl); substituted or unsubstituted C6-10 aryl; substituted or unsubstituted 5-10 membered heteroaryl; substituted or unsubstituted C3-10 cycloalkyl; substituted or unsubstituted C3-10 cycloalkenyl; substituted or unsubstituted 3-10 membered heterocycloalkyl; substituted or unsubstituted 3-7 membered heterocycloalkenyl; substituted or unsubstituted C6-10 aryl C1-6 alkyl; substituted or unsubstituted C1-6 alkyl C6-10 aryl; substituted or unsubstituted 5-10 membered heteroaryl C1-6 alkyl; and substituted or unsubstituted C1-6 alkyl 5-10 membered heteroaryl;

or two adjacent groups of R₃, R₄ and R₅ may be bonded to form a substituted or unsubstituted C6-10 aryl group, a substituted or unsubstituted 5-10 membered heteroaryl group, a substituted or unsubstituted C3-10 cycloalkyl group, or a substituted or unsubstituted 3-10 membered heterocycloalkyl group;

the “substituted” in “substituted or unsubstituted” means that being substituted by one or more substituents selected from the group consisting of H, hydroxyl, amino group, cyano, nitro, carboxyl, halogen, C1-6 alkyl, C1-6 haloalkyl or C1-6 hydroxyalkyl;

and meets one of the following conditions:

(1) when W, Z or T is substituted by one group of R₃, R₄ and R₅, the W, Z or T is N or CH;

(2) when W, Z or T is substituted by one group of R₃, R₄ and R₅ and this group is bonded to another group of R₃, R₄ and R₅ to form a substituted or unsubstituted C6-10 aryl or a substituted or unsubstituted 5-10 membered heteroaryl, the W, Z or T is C; for example, when W is substituted by R₃ and R₃ is bonded to the adjacent R₄ to form a substituted or unsubstituted C6-10 aryl or a substituted or unsubstituted 5-10 membered heteroaryl, the W is C;

(3) when W, Z or T is substituted by two of R₃, R₄ and R₅, the W, Z or T is C.

In a more specific embodiment of the present invention, in the general formula Ia,

X and Y are each independently selected from the group consisting of 0, S and NH;

U and V are each independently selected from the group consisting of C and S;

W, Z, and T are each independently selected from the group consisting of O, N, NRₐ, CO, C, CRₐ, and CH₂;

m is 0, 1 or 2; preferably 0 or 1;

n is 0, 1 or 2; preferably 0 or 1; and/or

R₁ is selected from H and deuterium.

In a more specific embodiment of the present invention, the general formula Ia is the general formula IIa as shown below:

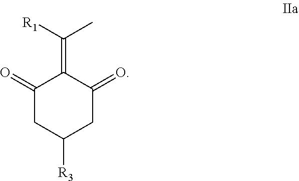

Wherein, R₁ is selected from the group consisting of H, deuterium, unsubstituted C1-6 alkyl or C1-6 alkyl substituted by a substituent selected from hydroxyl and halogen, and unsubstituted phenyl or phenyl substituted by a substituent selected from halogen, hydroxyl, C1-C6 alkyl and C1-C6 heteroalkyl;

R₃ is selected from the group consisting of H; hydroxyl; amino group; halogen; cyano; nitro; carboxyl; formyl; amide group; ester group; unsubstituted C1-6 alkyl or C1-6 alkyl substituted by a substituent selected from hydroxyl, halogen and C1-6 alkoxy; C1-6 heteroalkyl; C2-6 alkenyl; C2-6 alkynyl; substituted or unsubstituted —CONH₂—(C6-10 aryl); substituted or unsubstituted —CH═CH—(C6-10 aryl); substituted or unsubstituted C6-10 aryl; substituted or unsubstituted 5-10 membered heteroaryl; substituted or unsubstituted C3-10 cycloalkyl; substituted or unsubstituted C3-10 cycloalkenyl; substituted or unsubstituted 3-10 membered heterocycloalkyl; substituted or unsubstituted 3-7 membered heterocycloalkenyl; substituted or unsubstituted C6-10 aryl C1-6 alkyl; substituted or unsubstituted C1-6 alkyl C6-10 aryl; substituted or unsubstituted 5-10 membered heteroaryl C1-6 alkyl; and substituted or unsubstituted C1-6 alkyl 5-10 membered heteroaryl;
the “substituted” in “substituted or unsubstituted” means that being substituted by one or more substituents selected from the group consisting of H, hydroxyl, amino group, cyano, nitro, carboxyl, halogen, C1-6 alkyl, C1-6 haloalkyl or C1-6 hydroxyalkyl.

In a more specific embodiment of the present invention,

R₃ is selected from the following groups:

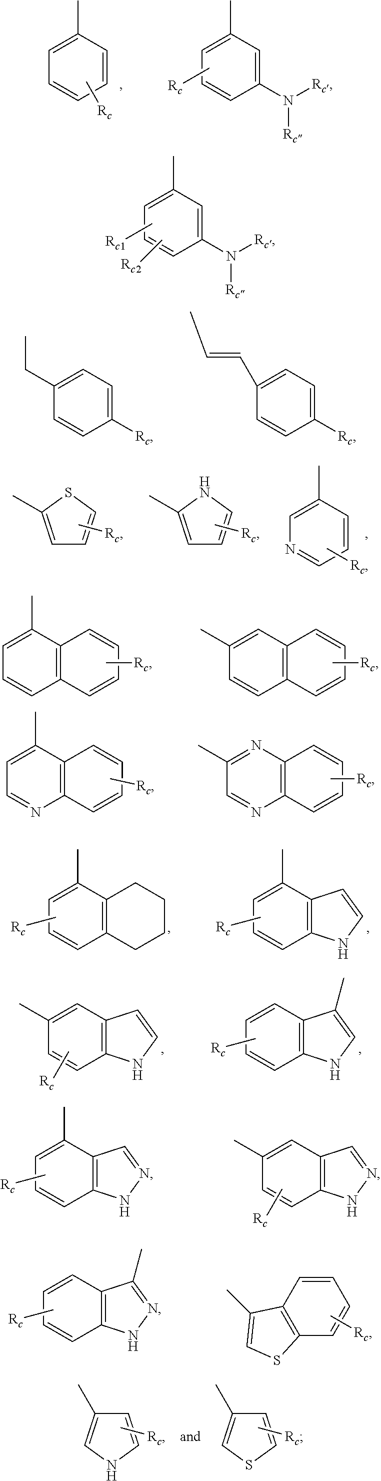

Wherein,

R_{c}, R_{c1}, R_{c2}, R_{c}′ and R_{c}″ are each independently selected from the group consisting of H, hydroxyl, amino group, NRaRa′, halogen, cyano, nitro, carboxyl, formyl, amide group, ester group, C1-6 haloalkyl, C1-6 hydroxyalkyl, C1-6 heteroalkyl, C1-6 alkoxy, C1-6 alkoxyalkyl, C2-6 alkenyl, C2-6 alkynyl, C6-10 aryl, 5-10 membered heteroaryl, C3-10 cycloalkyl, 3-10 membered heterocycloalkyl, 3-7 membered heterocycloalkenyl, C1-6 alkyl C6-10 aryl, 5-10 membered heteroaryl C1-6 alkyl or C1-6 alkyl 5-10 membered heteroaryl; preferably selected from the group consisting of H, hydroxyl, amino group, NRaRa′, halogen, carboxyl, formyl, amide group, ester group, C1-6 haloalkyl, C1-6 hydroxyalkyl, C1-6 heteroalkyl, C1-6 alkoxyl, C3-10 cycloalkyl, 3-10 membered heterocycloalkyl, substituted or unsubstituted phenyl or pyridyl;

Rₐ is H or C1-6 alkyl;

or R_{c1} and R_{c2} may be bonded to form C6-10 aryl, 5-10 membered heteroaryl, C3-10 cycloalkyl, or 3-10 membered heterocycloalkyl;

or R₃ is selected from the following groups:

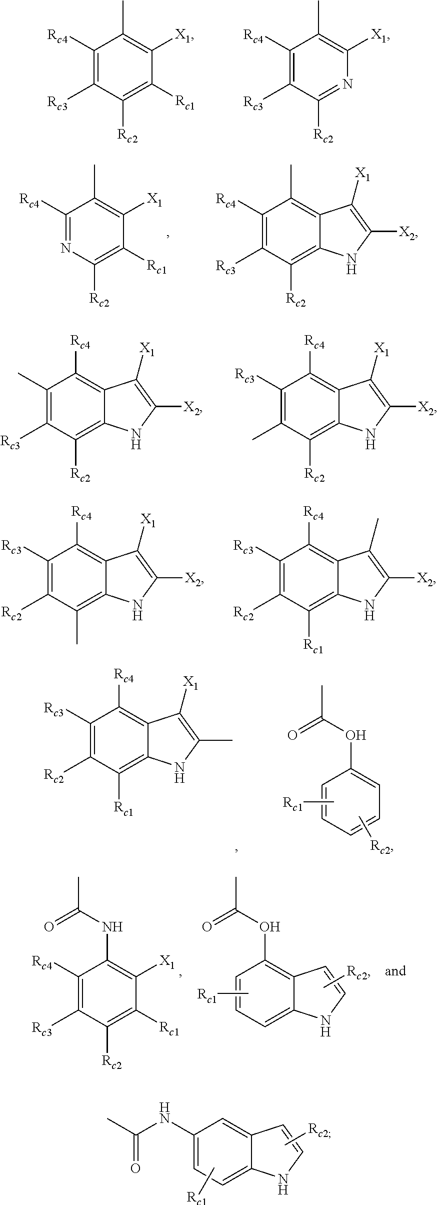

wherein, X₁ is F, Cl, Br, I or trifluoromethyl;

X₂ is H, F, Cl, Br, or I;

R_{c1}, R_{c2}, R_{c3} and R_{c4} are each independently selected from the group consisting of H, hydroxyl, amino group, NRaRa′, halogen, cyano, nitro, carboxyl, formyl, amide group, ester group, C1-6 haloalkyl, C1-6 hydroxyalkyl, C1-6 heteroalkyl, C1-6 alkoxy, C1-6 alkoxyalkyl, C2-6 alkenyl, C2-6 alkynyl, C6-10 aryl, 5-10 membered heteroaryl, C3-10 cycloalkyl, 3-10 membered heterocycloalkyl, 3-7 membered heterocycloalkenyl, C1-6 alkyl C6-10 aryl, 5-10 membered heteroaryl C1-6 alkyl and C1-6 alkyl 5-10 membered heteroaryl; preferably selected from the group consisting of H, hydroxyl, amino group, NRaRa′, halogen, carboxyl, formyl, amide group, ester group, C1-6 haloalkyl, C1-6 hydroxyalkyl, C1-6 heteroalkyl, C1-6 alkoxyl, C3-10 cycloalkyl, 3-10 membered heterocycloalkyl, substituted or unsubstituted phenyl or pyridyl;

Rₐ is H or C1-6 alkyl;

or R_{c1} and R_{c2}, or R_{c2} and R_{c3}, or R_{c3} and R_{c4} may be bonded to form C6-10 aryl, 5-10 membered heteroaryl, C3-10 cycloalkyl, and 3-10 membered heterocycloalkyl.

Data from the protein thermal shift assay will also indicate that the thermodynamic stability of proteins of the above mammalian ATG8 homologue covalent complexes is different from the thermodynamic stability of proteins of the mammalian ATG8 homologue. The covalent complex of the mammalian ATG8 homologue has a melting temperature that can be 2° C. or higher than the melting temperature of the mammalian ATG8 homologue. Preferably, the melting temperature of the above covalent complex maybe 5° C. higher than the melting temperature of LC3B.

In a more specific embodiment of the invention, the covalent complex of the mammalian ATG8 homologue has a melting temperature that is at least 2° C. higher than the mammalian ATG8 homologue, preferably at least 5° C. higher.

The thermodynamic stability of the covalent complex of the mammalian ATG8 homologue can be used to detect the covalent complex of the mammalian ATG8 homologue and can be used to diagnose and treat diseases associated with the mammalian ATG8 homologue.

The covalent complex of the mammalian ATG8 homologue can play a role in diagnosing and treating diseases associated with the mammalian ATG8 homologue. For example, this covalent complex can be used as a biomarker for diagnosing and treating diseases associated with the mammalian ATG8 homologue.

Therefore, the present invention also provides use of the covalent complex of the mammalian ATG8 homologue in manufacturing a reagent for diagnosing and treating a disease which is selected from the group consisting of a tumor, a cardiovascular disease, an autoimmune disease, a neurodegenerative disease, hypertension, bone tissues and bone-related diseases, Crohn's disease, acute kidney injury, cerebral ischemia, retinal diseases, bronchial asthma, Vici syndrome, and infectious diseases. Said tumor is selected from the group consisting of liver cancer, lung cancer, pancreatic cancer, breast cancer, cervical cancer, endometrial cancer, colorectal cancer, gastric cancer, lung cancer, nasopharyngeal cancer, ovarian cancer, prostate cancer, leukemia, lymphoma, myeloma.

The present invention also provides a method for diagnosing and treating a disease by using the covalent complex of the mammalian ATG8 homologue, in which the disease is selected from the group consisting of a tumor, a cardiovascular disease, an autoimmune disease, a neurodegenerative disease, hypertension, bone tissues and bone-related diseases, Crohn's disease, acute kidney injury, cerebral ischemia, retinal diseases, bronchial asthma, Vici syndrome, and infectious diseases. Said tumor is selected from the group consisting of liver cancer, lung cancer, pancreatic cancer, breast cancer, cervical cancer, endometrial cancer, colorectal cancer, gastric cancer, lung cancer, nasopharyngeal cancer, ovarian cancer, prostate cancer, leukemia, lymphoma, myeloma.

### BRIEF DESCRIPTION OF THE DRAWINGS

FIG. 1 shows a covalent complex of Compound A-LC3B, in which the interaction of the modified lysine at position 49 with the surrounding amino acids has been illustrated (black dotted line; the unit of distance is angstrom).

FIG. 2 shows the selectivity of Compound A for the lysine at position 49 in LC3B.

FIG. 3 shows the conservative first lysine at positions 46-55 in the superimposed LC3A, LC3B, LC3C, GABARAP, GABARAPL1 and GABARAPL2 (PDB ID: 3WAL, 3VTU, 3WAM, 1GNU, 2R2Q and 4CO7).

FIG. 4 is a mass spectrum demonstrating the lysine at position 49 of LC3B being covalently modified by Compound B, (A) reaction mechanism; (B) ions of b type and y type.

FIG. 5 is a mass spectrum demonstrating the lysine at position 49 of LC3B being covalently modified by Compound C, (A) reaction mechanism; (B) ions of b type and y type.

FIG. 6 is a mass spectrum demonstrating the lysine at position 49 of LC3B being covalently modified by Compound D, (A) reaction mechanism; (B) ions of b type and y type.

FIG. 7 shows the effect of Compound B on autophagy, (A) Immunoblotting assay of LC3-I/LC3-II protein; (B) Immunofluorescence staining and fluorescence microscopy.

### DETAILED DESCRIPTION OF THE INVENTION

Further details of the invention will be described in the following part. Various improvements and modifications would be obviously made by those skilled in the art in accordance with the present invention without departing from the spirit and scope of the invention, which should be covered within the protection scope limited by the appended claims of the invention.

The present invention provides a method for modulating a mammalian ATG8 homologue, comprising: providing a compound SM-LG including a moiety SM- having a function of modulating a mammalian ATG8 homologue and a leaving moiety -LG; the compound SM-LG reacts with a mammalian ATG8 homologue to produce a covalent complex of the mammalian ATG8 homologue. The leaving moiety -LG is bonded to a hydrogen ion to form a small molecule compound LG-H. The method reflects recent advancements in treating some related diseases by using a mammalian ATG8 homologue as a target.

Compounds having activity for LC3B are screened by fluorescence polarization (FP) assay (which is described in detail hereinafter in this application) to give one type of compounds SM-LG, wherein SM- is a moiety having function for modulating a mammalian ATG8 homologue, -LG is a moiety that leaves during the reaction with the mammalian ATG8 homologue. Such types of compounds have a time-dependent inhibition against LC3B.

In a more specific embodiment of the present invention, the SM- moiety in the compound SM-LG is defined as above.

In a more specific embodiment of the present invention, the -LG in the compound SM-LG represents -J-K-M-Q, wherein,

J is NRₐ, NORₐ, O, S or

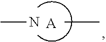

wherein

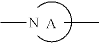

is a 3-10 membered heterocycloalkylene group containing at least one nitrogen atom or a 3-7 membered heterocycloalkenylene group containing at least one nitrogen atom;

K is a covalent bond, NRₐ, CR_{c}R_{c′} or CR_{c}R_{c′}CR_{c}R_{c′};

M is a covalent bond, CR_{c}R_{c′}, a 3-10 membered heterocycloalkylene group, a 3-7 membered heterocycloalkenylene group or a 5-10 membered heteroarylene group;

Q is hydrogen, C1-6 alkyl, C1-6 hydroxyalkyl, —(CH₂)ₚ—C(O)R_{b}, —(CH₂)ₚ—C(O)NHR_{b}, —(CH₂)ₚ—C(S)R_{b}, —(CH₂)ₚ—C(S)NHR_{b}, —(CH₂)ₚ—SO₂R_{b} or —(CH₂)ₚ—SO₂NHR_{b},

wherein,

p is 0, 1, 2 or 3; preferably 0, 1 or 2, more preferably 0 or 1;

each R_{b} is independently C1-6 alkyl, C2-6 alkenyl, NHRₐ, NRₐRₐ′, substituted or unsubstituted phenyl, or substituted or unsubstituted 3-7 membered heterocyclic group,

Rₐ and Rₐ′ are each independently H or C1-6 alkyl;

R_{c} and R_{c}′ are each independently selected from the group consisting of H, hydroxyl, amino group, cyano, nitro, carboxyl, halogen, C1-6 alkyl, C1-6 haloalkyl and C1-6 hydroxy alkyl.

When -LG is -J-K-M-Q, after the covalent compound is formed, LG-H becomes H-J-K-M-Q. And the H-J-K-M-Q is the small molecule compound referred herein.

In a more specific embodiment of the invention, -LG represents —OH in the compound SM-LG.

When -LG is —OH, after the covalent compound is formed, LG-H becomes H₂O (a water molecule).

In a more specific embodiment of the invention, LG is selected from the following groups:

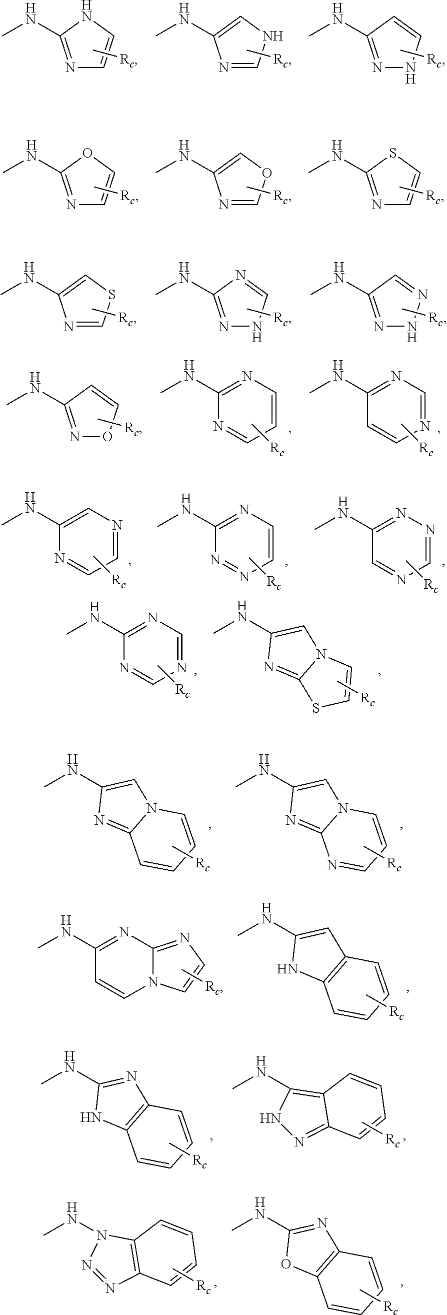

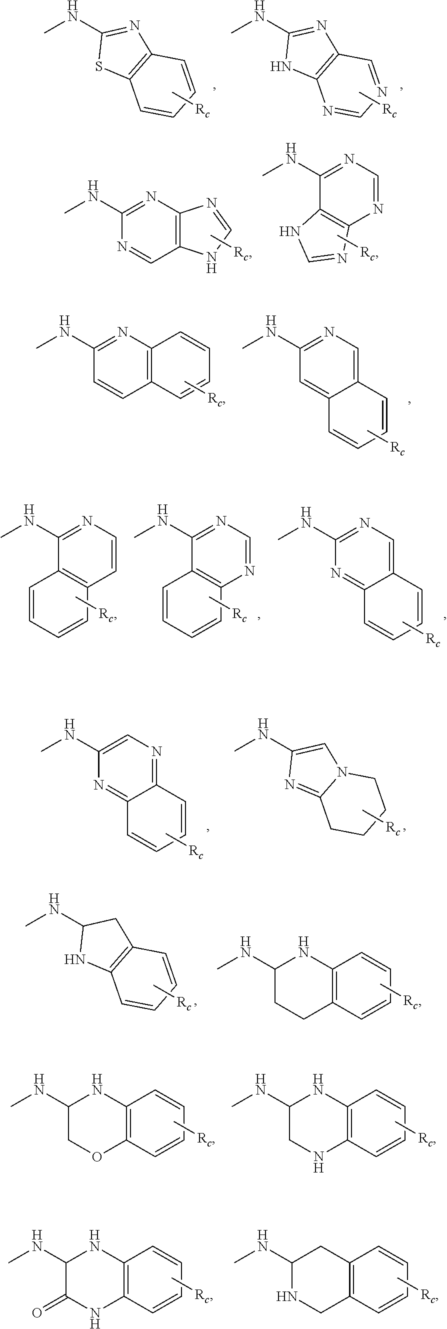

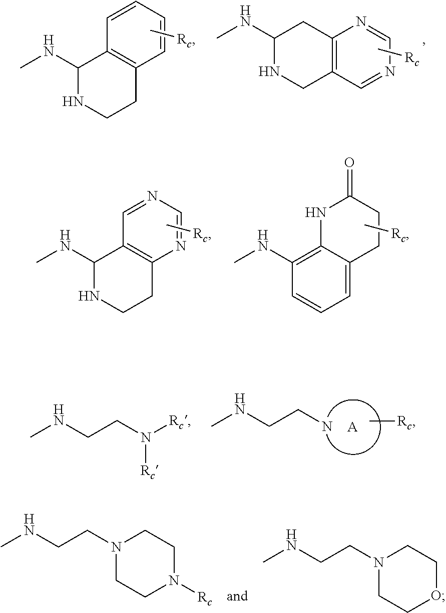

wherein,

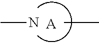

is a 3-10 membered heterocycloalkylene group containing at least one nitrogen atom or a 3-7 membered heterocycloalkenylene group containing at least one nitrogen atom;

R_{c}, R_{c}′ and R_{c}″ are each independently selected from the group consisting of H, hydroxyl, amino group, cyano, nitro, carboxyl, halogen, C1-6 alkyl, C1-6 haloalkyl and C1-6 hydroxy alkyl.

In a more specific embodiment of the present invention, -LG is selected from the following groups:

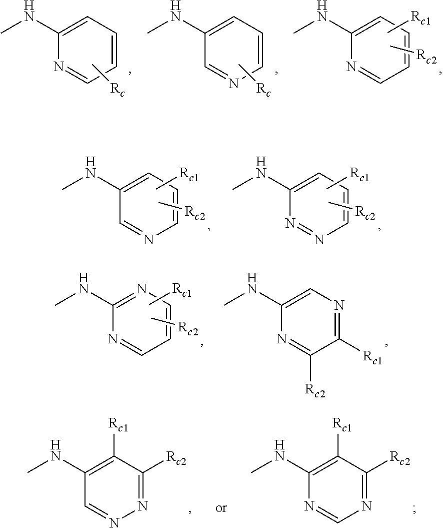

wherein,

R_{c} is selected from the group consisting of H, hydroxyl, amino group, cyano, nitro, carboxyl, halogen, C1-6 alkyl, C1-6 haloalkyl or C1-6 hydroxyalkyl;

R_{c1} and R_{c2} may be bonded to form C6-10 aryl, 5-10 membered heteroaryl, C3-10 cycloalkyl, or 3-10 membered heterocycloalkyl.

The terms used in the present invention have their ordinary meanings in the art, and in case of conflict, it is applicable to the definitions used in this application. Chemical names, common names and chemical structures may be used interchangeably to describe that same structure. These definitions apply regardless of whether a term is used by itself or in combination with other terms. Thus, the definition of “C1-6 alkyl” is applicable to “C1-6 alkyl” as well as the “C1-6 alkyl” portion of “C1-6 hydroxyalkyl”, “C1-6 haloalkyl”, “C6-10 aryl C1-6 alkyl”, “C1-6 alkyl C6-10 aryl”, “C1-6 alkoxy” and the like.

“Halogen” (or “halo”) refers to fluorine, chlorine, bromine, or iodine.

“C1-6 alkyl” refers to a straight or branched alkyl group having 1 to 6 carbon atoms, preferably a straight or branched alkyl group having 1 to 4 carbon atoms. “Branched” refers to one or more alkyl groups, such as methyl, ethyl or propyl and the like, is connected to a straight alkyl group. Preferred C1-6 alkyl group includes, but not limited to, methyl, ethyl, n-propyl, isopropyl, n-butyl, i-butyl, and t-butyl and the like.

“C1-6 haloalkyl” refers to a C1-6 alkyl as defined above having one or more halo group substituent(s).

“C1-6 heteroalkyl” refers to a C1-6 alkyl as defined above having one or more substituent(s) selected from the group consisting of O, S, N, —(S═O)—, —(O═S═O)—, etc.

“C2-6 alkenyl” refers to a straight or branched alkenyl group having 2 to 6 carbon atoms, preferably 2 to 4 carbon atoms. “Branched” refers to one or more lower C1-6 alkyl group is connected to a straight C2-6 alkenyl group chain. Preferred C2-6 alkenyl group includes, but not limited to, ethenyl, propenyl, n-butenyl, 3-methylbutenyl, n-pentenyl and the like.

“C1-6 alkylene” refers to a bivalent group obtained by removal of a hydrogen atom from a C1-6 alkyl group as defined above. Preferred C1-6 alkylene group includes, but not limited to, methylene, ethylidene and propylidene, etc. Generally, it can be optionally and equivalently expressed herein as —(C1-6 alkyl)-, for example —CH₂CH₂— is an ethylidene.

“C2-6 alkynyl” refers to a straight or branched alkynyl group having 2 to 6 carbon atoms, preferably 2 to 6 carbon atoms, more preferably having 2 to 4 carbon atoms. “Branched” refers to one or more alkyl group having 2 to 4 carbon atoms is connected to a straight alkynyl group chain. Preferred C2-6 alkynyl group includes, but not limited to, ethynyl, propynyl, 2-butynyl and 3-methylbutynyl, etc.

“C2-6 alkenylene” refers to a difunctional group obtained by removal of hydrogen from a C2-6 alkenyl group as defined above. Preferred C2-6 alkenylene group includes, but not limited to, —CH═CH—, —C(CH₃)═CH—, —CH═CHCH₂—, etc.

“C6-10 aryl” refers to an aromatic monocyclic or multicyclic ring system having 6 to 10 carbon atoms. Preferably, the C6-10 aryl group includes, but not limited to, phenyl and naphthyl.

“C6-10 arylidene” refers to a bivalent group obtained by removing a hydrogen atom from a C6-10 aryl group as defined above, for example

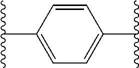 [SMILES: *c1ccc(*)cc1]

is p-phenylene.

“5-10 membered heteroaryl” refers to an aromatic monocyclic or multicyclic ring group having 5 to 10 ring atoms. The 5-10 membered heteroaryl group includes 1 to 4 hetero atoms selected from N, O and S. Preferred 5-10 membered heteroaryl group includes 5 to 6 ring atoms. The nitrogen atom of the 5-10 membered heteroaryl group can be optionally oxidized to the corresponding N-oxide. Preferred 5-10 membered heteroaryl group includes, but not limited to, pyridyl, pyrazinyl, furanyl, thienyl, pyrimidinyl, pyridone, oxazolyl, isothiazolyl, oxazolyl, oxadiazolyl, thiazolyl, thiadiazolyl, pyrazolyl, furazanyl, pyrrolyl, triazolyl, 1,2,4-thiadiazolyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the oxides thereof and the like. The term “5-10 membered heteroaryl” also refers to partially saturated 5-10 membered heteroaryl group, such as, for example, tetrahydroisoquinolyl, tetrahydroquinolyl and the like.

“C3-10 cycloalkyl” refers to a non-aromatic monocyclic or multicyclic ring group having 3 to 10 carbon atoms, preferably 3 to 6 carbon atoms. Preferred monocyclic C3-10 cycloalkyl includes, but not limited to, cyclopropyl, cyclopentyl, cyclohexyl, cycloheptyl and the like. Preferred multicyclic cycloalkyl includes, but not limited to, [1.1.1]-bicyclopentane, 1-capryl, norbornyl, adamantyl and the like.

“C3-10 cycloalkenyl” refers to a non-aromatic monocyclic or multicyclic ring group having 3 to 10 carbon atoms on the ring, which contains at least one carbon-carbon double bond within the ring. Preferably, it has 3 to 7 carbon atoms on the ring, most preferably having 5 to 7 carbon atoms on the ring. Preferred cycloalkenyl includes, but not limited to, cyclopropenyl, cyclobutenyl, cyclopentenyl, cyclohexenyl, cyclohetpenyl, cycloheptane-1,3-dienyl, norbomylenyl and the like.

“3-10 membered heterocycloalkyl” or “3-10 membered heterocyclyl” refers to a non-aromatic monocyclic or multicyclic ring group having 3 to 10 ring atoms, preferably 5 to 10 ring atoms, more preferably 5 to 6 ring atoms, in which the 3-10 membered heterocyclyl group includes 1 to 4 hetero atoms selected from N, O and S. The nitrogen or sulfur atom of the 3-10 membered heterocyclyl can be optionally oxidized to the corresponding N-oxide, S-oxide or S-dioxide. Thus, the term “oxide” of the invention refers to the corresponding N-oxide, S-oxide, or S-dioxide. “3-10 membered heterocyclyl” also includes rings in which two available hydrogens on the same carbon atom are simultaneously replaced by one single group ═O (for example, a carbonyl group). Such ═O group may be referred as “oxo-” in the present invention. Preferred monocyclic 3-10 membered heterocycloalkyl includes, but not limited to, piperidyl, oxetanyl, pyrrolyl, piperazinyl, morpholinyl, thiomorpholinyl, thiazolidinyl, 1,4-dioxin alkyl, tetrahydrofuranyl, tetrahydrothiophenyl, lactamyl (such as pyrrolidinone), lactone group having 3 to 10 ring atoms and oxides thereof.

“3-7 membered heterocycloalkenyl” refers to a non-aromatic monocyclic or multicyclic ring group having 3 to 7 ring atoms, preferably 5 to 6 ring atoms, in which the 3-7 membered heterocycloalkenyl group includes 1 to 4 hetero atoms selected from N, O and S, and includes at least one carbon-carbon double bond or carbon-nitrogen double bond. The aza, oxa or thia contained in the group name refers to at least one nitrogen, oxygen or sulfur atom respectively presented as a ring atom. The nitrogen or sulfur atom in the 3-7 membered heterocyclenyl group can be optionally oxidized to the corresponding N-oxide, S-oxide or S-dioxide. Preferred 3-7 membered heterocyclenyl group includes, but not limited to, 1,2,3,4-tetrahydropyridinyl, 1,2-dihydropyridinyl, 1,4-dihydropyridinyl, 1,2,3,6-tetrahydropyridinyl, 1,4,5,6-tetrahydropyrimidinyl, 2-pyrrolinyl, 3-pyrrolinyl, 2-imidazolinyl, 2-pyrazolinyl, dihydroimidazolyl, dihydrooxazolyl, dihydrooxadiazolyl, dihydrothiazolyl, 3,4-dihydro-2H-pyranyl, dihydrofuranyl, fluorodihydrofuranyl, and the oxides thereof, and the like. “3-7 membered heterocyclenyl” may also be rings in which two available hydrogens on the same carbon atom are simultaneously replaced by one single group ═O (i.e., forming a carbonyl).

“C6-10 aryl C1-6 alkyl” refers to a group formed by replacing one hydrogen on the C6-10 alkyl as defined above by the C1-6 aryl as defined above. Preferred C6-10 aryl C1-6 alkyl includes, but not limited to, benzyl, 2-phenethyl and naphthalenylmethyl. The C6-10 aryl C1-6 alkyl is bonded to the parent moiety by a C1-6 alkyl group. Similarly, “5-10 membered heteroaryl C1-6 alkyl”, “C3-10 cycloalkyl C1-6 alkyl”, “C3-10 cycloalkenyl C1-6 alkyl”, “3-10 membered heterocycloalkyl C1-6 alkyl”, “3-7 membered heterocycloalkenyl C1-6 alkyl” and the like refer to the 5-10 membered heteroaryl, C3-10 cycloalkyl, C3-10 cycloalkenyl, 3-10 membered heterocycloalkyl, 3-7 membered heterocycloalkenyl and the like as defined herein are bonded to the parent moiety by a C1-6 alkyl group.

“C1-6 aryl C6-10 alkyl” refers to a group formed by replacing one hydrogen on the C6-10 aryl as defined above by the C1-6 alkyl as defined above. Preferred C1-6 alkyl C6-10 aryl includes, but not limited to, tolyl. The C1-6 alkyl C6-10 aryl is boned to the parent moiety by a C6-10 aryl group.

“5-10 membered heteroaryl C1-6 alkyl” refers to a group formed by replacing one hydrogen on the C1-6 alkyl as defined above by the 5-10 membered heteroaryl as defined above. Preferred 5-10 membered heteroaryl C1-6 alkyl includes, but not limited to, pyridylmethyl and quinolin-3-ylmethyl. The 5-10 membered heteroaryl C1-6 alkyl is boned to the parent moiety by a C1-6 alkyl group.

“C1-6 hydroxyalkyl” refers to a hydroxyl-substituted C1-6 alkyl group, wherein the C1-6 alkyl group is described as above. Preferred C1-6 hydroxyalkyl includes, but not limited to, hydroxymethyl and 2-hydroxyethyl.

“C1-6 alkoxy” refers to a C1-6 alkyl-O— group, wherein the C1-6 alkyl group is described as above. Preferred C1-6 alkoxy includes, but not limited to, methoxy, ethoxy, n-propoxy, isopropoxy and n-butoxy, which is bonded to the parent moiety by —O—. “C1-6 alkyoxyalkyl” refers to a group derived from a C1-6 alkoxy and C1-6 alkyl as defined herein, which is bonded to the parent moiety by a C1-6 alkyl group.

“Ester group” refers to a group that is obtained by removing one hydrogen atom from an ester formed by esterification of an aliphatic or aromatic carboxylic acid having 1 to 20 carbon atoms with a primary, secondary or tertiary alcohol having 1 to 20 carbon atoms. Preferred ester group includes, but not limited to, methoxycarbonyl, ethoxycarbonyl, isopropyl ester group, tert-butyl ester group, phenyl ester group.

“Amide group” refers to a group that is obtained by removing one hydrogen atom from an amide formed by amidation of an aliphatic or aromatic carboxylic acid having 1 to 20 carbon atoms with a primary or secondary amine having 1 to 20 carbon atoms.

“Heterocycloalkylene”, “heterocycloalkenyl” or “heteroarylene” refers to a bivalent group formed by missing one hydrogen atom from the corresponding heterocycloalkyl, heterocycloalkenyl or heteroaryl group as described above.

Any of the foregoing functional groups may be unsubstituted or substituted as described herein. The term “substituted” (or substitute) refers to that one or more hydrogens on the designated atom is replaced with a group selected from the indicated groups, provided that not exceeding the designated atom's normal valency and the substitution forms a stable compound. Combinations of substituents and/or variables are permissible only if such combinations result in stable compounds. “Stable compound” or “stable structure” is a compound having sufficient stability that can be separated into a useful purity from a reaction mixture and can be formulated to an efficacious therapeutic agent.

The term “substituted” refers to a particular group that is unsubstituted or substituted with one or more substituents. Substituents include, but not limited to, hydrogen, hydroxyl, amino, cyano, nitro, carboxy, halo, C1-6 alkyl, C1-6 haloalkyl or C1-6 hydroxyalkyl. Two adjacent substituents may be joined to form a C6-10 aryl group, a 5-10 membered heteroaryl group, a C3-10 cycloalkyl group or a 3-10 membered heterocycloalkyl group. The substitution on C6-10 aryl, 5-10 membered heteroaryl, C3-10 cycloalkyl, 3-10 membered heterocycloalkyl, 3-7 membered heterocycloalkenyl groups and the like includes, but not limited to, substitution in any ring portion of the groups.

In the present application, if a group is a “covalent bond”, it means that the group “does not exist” and the two linked groups are joined by a covalent bond. For example, in the substituent “-J-K-M-Q”, if K is a covalent bond, then this substituent becomes “-J-M-Q”.

The above compound SM-LG can be prepared by various methods known in the art, and the following reaction scheme is an optional solution for a general reaction procedure for preparing the above compounds:

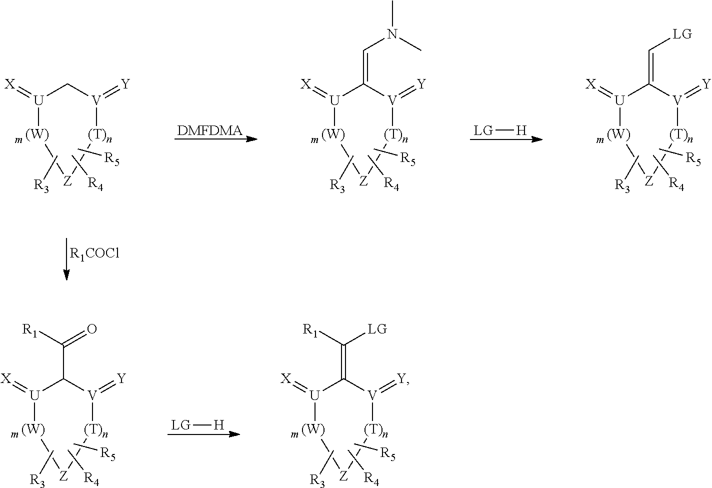

wherein each group is defined as the above, and DMFDMA is N,N-dimethylformamide dimethyl acetal.

The compound SM-LG can be prepared by methods described in some references known to those of ordinary skill in the art. These references include, for example, Bioorganic & Medicinal Chemistry Letters, 24(16), 3764-3771, 2014; Chemistry—A European Journal, 20(9), 2445-2448, 2014; Bioorganic & Medicinal Chemistry, 20(2), 1029-1045, 2012; Journal of Organic Chemistry, 82(5), 2630-2640, 2017; Tetrahedron Letters, 49 (2008), 4725-4727; Journal of Organic Chemistry, 78(9), 4563-4567, 2013; Heterocycles, 28(2), 1015-35, 1989; Journal of Medicinal Chemistry, 57(10), 3924-3938, 2014; Journal of Organic Chemistry, 66(24), 8000-8009, 2001; and Tetrahedron Letters, 56(45), 6287-6289, 2015.

The IC₅₀ values of some SM-LG compounds for inhibiting the activity of LC3B have been recorded in the patent applications submitted to the National Intellectual Property Administration of China on the same day by the same applicant, titled by “Compounds used as autophagy modulators and a method for preparing and using the same” and “Isoindolone-imide ring-1,3-dione-2-ene compound, composition and use thereof,” respectively, both of which are incorporated herein by reference in their entirety.

### Examples

The invention is further illustrated below in conjunction with various embodiments. Examples of the embodiments are illustrated in the accompanying drawings. It is to be understood that all these examples are not intended to limit the scope of the invention. Many other embodiments can be enclosed within the present invention, and various improvements and modifications would be obviously made by those skilled in the art in accordance with the present invention without departing from the spirit and scope of the invention, which should be covered within the protection scope limited by the appended claims of the invention.

Those skilled in the art will readily appreciate that these compounds can be prepared by known variations with the conditions and procedures used in the following preparation methods.

The starting reactants used in the present invention are all commercially available unless otherwise specified.

LC3B is the most-studied object of the mammalian ATG8 homologues, and it is a marker of autophagy in mammalian cells. In the present application, “LC3B”, “MAP1LC3B”, and “microtubule-associated protein 1 light chain 313” are all used to describe the same protein. The protein sequence of LC3B used in the examples of the present application are as follows:

| Proteins | Sequences | Assays |
|---|---|---|
| LC3B | SEQ ID NO: 1 | Full-length protein template |
| GST-LC3B | SEQ ID NO: 2 | Fluorescence Polarization Assay |
| LC3B (1-125) | SEQ ID NO: 3 | Protein Thermal Shift Assay, Mass |
| | | Spectrometry |
| LC3B (2-119) | SEQ ID NO: 4 | Crystal Complex Structure and Analysis |
| N-terminal | SEQ ID NO: 5 | Fluorescence Polarization Assay |
| FITC-labeled | | |
| peptide | | |
| LBP2 | SEQ ID NO: 6 | Protein Thermal Shift Assay |
| K8A | SEQ ID NO: 7 | Protein Thermal Shift Assay |
| K30A | SEQ ID NO: 8 | Protein Thermal Shift Assay |
| K39A | SEQ ID NO: 9 | Protein Thermal Shift Assay |
| K42A | SEQ ID NO: 10 | Protein Thermal Shift Assay |
| K49A | SEQ ID NO: 11 | Protein Thermal Shift Assay |
| K51A | SEQ ID NO: 12 | Protein Thermal Shift Assay |
| K65A | SEQ ID NO: 13 | Protein Thermal Shift Assay |
| K103A | SEQ ID NO: 14 | Protein Thermal Shift Assay |
| K122A | SEQ ID NO: 15 | Protein Thermal Shift Assay |

### Vector Construction

The cDNA encoding human LC3B (SEQ ID NO: 1) was purchased from Addgene (NCBI Accession No. NP_073729.1). The template was PCR amplified and constructed into plasmid expression vector pGEX-6P-1 or pGEX-4T-1, respectively.

### Protein Expression and Purification

GST-LC3B (1-125) (SEQ ID NO: 2) and each mutant protein were expressed by IPTG induction in DE3 competent cells at 16° C. After collecting and resuspending DE3 competent cells, the cells were sonicated and centrifuged. The resulted supernatant was hanged on the column (GSTrap FF, GE), followed by eluting with glutathione-containing eluant, and then the target protein was produced by gel filtration chromatography. This fusion protein was used directly in the fluorescence polarization assay. The proteins used in the protein thermal shift assay, mass spectrometry, and protein crystallization experiments were produced by PP enzyme or thrombin digestion and the following gel filtration chromatography to the GST-fusion protein obtained by the above method.

### Testing Experiment by Fluorescence Polarization (FP) Assay

Recombinant protein GST-LC3B (final concentration 180 nM) (SEQ ID NO: 2) and N-terminal FITC-labeled peptide (SEQ ID NO: 5, final concentration 18 nM) were placed in FP buffer solution (50 mM HEPES pH 7.5, 0.1 mg/ml BSA and 1 mM DTT), into which the compound gradually diluted by the FP buffer was added, then the resulted mixture was incubated in dark at 25° C. Fluorescence polarization values were monitored (PerkinElmer Envision, the wavelength of the emission light, 480 nm; the wavelength of the absorption light, 535 nm) and the IC50 values were calculated by the GraphPad Prism 6.0 program.

Representation of the IC50 value of the compounds: “10004<IC50≤1 mM” is considered as having low activity (+) against LC3B. “15 μM<<IC50≤100 μM” of the compound is considered as having moderate activity (++) against LC3B. “3 μM<IC50≤15 μM” is considered as having high activity (+++) against LC3B. “IC50≤3 μM” is considered as having higher activity (++++) against LC3B.

### Protein Thermal Shift Assay

The protein thermal shift assay is used to test the effect of compounds on the thermodynamic stability of proteins via the Quant Studio 6 Flex Real-Time PCR system. Protein LC3B (1-125) (SEQ ID NO: 3) and each mutant protein (SEQ ID NO: 7-15) (final concentration 4 μM), environmentally sensitive dye (5×SYPRO orange, Invitrogen) and compound (final concentration 40 μM) were mixed in the buffer solution (50 mM HEPES pH 7.5, 1 mM DTT) to a total volume of 20 μL. The sample was heated from 25° C. to 95° C. with a heating rate of 3%. Changes in fluorescence intensity were monitored and each melting temperature (Tm), represented by AT in the unit of ° C., was calculated by Protein Thermal Shift Software 1.1 (ABI). Data of the fluorescence polarization assay and protein thermal shift assay for partial compounds were listed in table 1.

TABLE 1
Data of the fluorescence polarization assay and protein thermal shift assay

| | | | | IC₅₀ | ΔT |
|---|---|---|---|---|---|
| Compound | Structure | Name | ¹HNMR | (μM) | (° C.) |
| A | 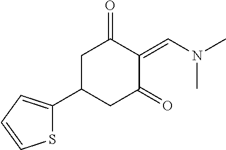 [SMILES: CN(C)C=C1C(=O)CC(c2cccs2)CC1=O] | 2-((Dimethyl amino)methyl- ene)-5-(tlhio- phen-2-yl)cyclo- hexane-1,3- dione | (400 MHz, CDCl₃) δ 8.08 (s, 1H), 7.19 (d, J = 5.1 Hz, 1H), 6.99- 6.92 (m, 1H), 6.88 (d, J = 3.4 Hz, 1H), 3.71-3.59 (m, 1H), 3.43 (s, 3H), 3.22 (s, 3H), 2.92 (m, 2H), 2.74 (m, 2H). | 1.56 | 10.37 |
| B | 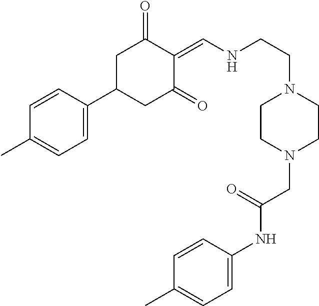 [SMILES: Cc1ccc(NC(=O)CN2CCN(CCNC=C3C(=O)CC(c4ccc(C)cc4)CC3=O)CC2)cc1] | 2-(4-(2-((2,6- dioxo-4-(p-tol yl)cyclohexyl) methyl)amino) ethyl)piperazin- 1-yl)-N-(p- tolyl)acetamide | (400 MHz, CD₃OD) δ 8.24 (s, 1H), 7.43 (d, J = 8.5 Hz, 2H), 7.14 (m, 6H), 3.61 (t, J = 5.8 Hz, 2H), 3.16 (s, 2H), 2.68 (m, 14H), 2.29 (s, 6H). | 3.09 | 10.55 |
| C | 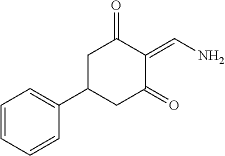 [SMILES: NC=C1C(=O)CC(c2ccccc2)CC1=O] | 2-(aminomethyl- ene)-5-phenyl- cyclohexane- 1,3-dione | (300 MHz, CDCl₃) δ 10.53 (s, 1H), 8.27 (dd, J = 15.7, 8.9 Hz, 1H), 7.35 (t, J = 7.4 Hz, 2H), 7.23 (d, J = 7.7 Hz, 3H), 6.69 (s, 2H), 3.36 (s, 1H), 2.83-2.60 (m, 4H). | 28.77 | 2.87 |
| D | 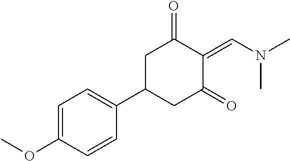 | 2-((Dimethyl amino)methyl- ene)-5-(4- methoxyphenyl) cyclohexane-1, 3-dione | (300 MHz, CDCl₃) δ 8.04 (d, J = 16.8 Hz, 1H), 7.16 (d, J = 8.4 Hz, 2H), 6.86 (d, J = 8.5 Hz, 2H), 3.79 (s, 3H), 3.41 (s, 3H), 3.30 (m, 1H), 3.19 (s, 3H), 2.69 (m, 4H). | 1.46 | 10.82 |
| E | 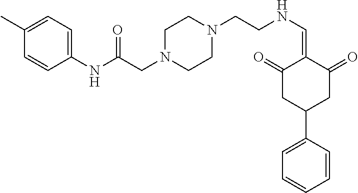 [SMILES: Cc1ccc(NC(=O)CN2CCN(CCNC=C3C(=O)CC(c4ccccc4)CC3=O)CC2)cc1] | 5-phenyl-2- (((2-(4-(2-p-tol uidine acetyl) piperazin-1-yl) ethyl)amino) methylene) cyclohexane-1,3- dione | ¹H NMR (400 MHz, dmso) δ 11.05- 10.91 (m, 1H), 9.59 (s, 1H), 8.14 (d, J = 14.7 Hz, 1H), 7.50 (d, J = 8.4 Hz, 2H), 7.33-7.29 (m, 4H), 7.25-7.19 (m, 1H), 7.11 (d, J = 8.4 Hz, 2H), 3.62-3.54 (m, 2H), 3.33-3.26 (m, 1H), 3.09 (s, 2H), 2.80-2.64 (m, 2H), 2.57-2.45 (m, 12H), 2.25 (s, 3H); MS: 475.2 [M + H]. | 5.0 | 10.21 |
| F | 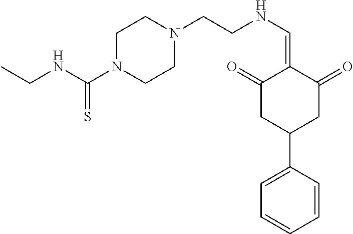 | 5-phenyl-2- (((2-(4-(ethyl- amino thiocarbonyl) piperazin-1-yl) ethyl)amino) methylene) cyclohexane-1,3- dione | ¹H NMR (400 MHz, dmso) δ 11.00- 10.90 (m, 1H), 7.48 (s, 1H), 8.12 (d, J = 14.7 Hz, 1H), 7.35-7.25 (m, 4H), 7.24-7.19 (m, 1H), 4.20 (q, 2H), 3.71-3.68 (m, 6H), 3.30-3.22 (m, 1H), 2.75-2.60 (m, 2H), 2.60- 2.54 (m, 8H), 1.32 (t, 3H); MS: 475.2 [M + H]. | 6.54 | 10.51 |
| G | 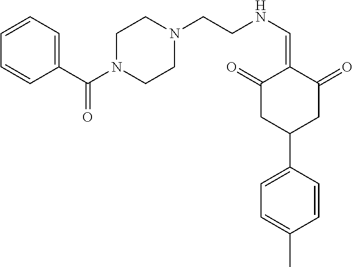 | 5-phenyl-2- (((2-(4-(benzoyl) piperazin-1-yl) ethyl)amino) methylene) cyclohexane-1,3- dione | ¹H NMR (400 MHz, dmso) δ 11.03- 10.95 (m, 1H), 8.12 (d, J = 14.6 Hz, 1H), 7.38-7.30 (m, 4H), 7.26-7.17 (m, 3H), 7.14 (d, J = 8.0 Hz, 2H), 3.75-3.70 (m, 6H), 3.35-3.30 (m, 1H), 2.80-2.64 (m, 2H), 2.59-2.56 (m, 8H); MS: 415.2 [M + H]. | 4.83 | 9.83 |
| H | 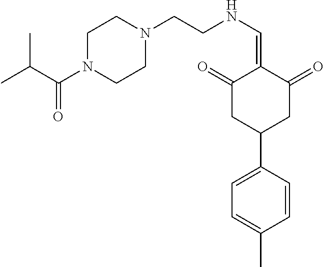 [SMILES: Cc1ccc(C2CC(=O)C(=CNCCN3CCN(C(=O)C(C)C)CC3)C(=O)C2)cc1] | 5-phenyl-2- (((2-(4-(2-methyl- propionyl) piperazin-1-yl) ethyl)amino) methylene) cyclohexane-1,3- dione | ¹H NMR (400 MHz, dmso) δ 11.03- 10.95 (m, 1H), 8.12 (d, J = 14.6 Hz, 1H), 7.22 (d, J = 8.1 Hz, 2H), 7.14 (d, J = 8.0 Hz, 2H), 3.75-3.70 (m, 6H), 3.35-3.30 (m, 1H), 2.80-2.64 (m, 3H), 2.59-2.56 (m, 8H), 2.27 (s, 3H), 1.10 (d, 6H); MS: 412.2 [M + H]. | 5.00 | 10.21 |
| I | 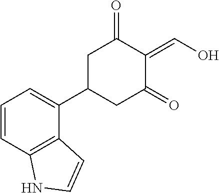 [SMILES: O=C1CC(c2cccc3[nH]ccc23)CC(=O)C1=CO] | 2-(hydroxy- methylene)- 5-(1H-indol-4- yl)cyclohexane- 1,3-dione | ¹H NMR (400 MHz, DMSO-d₆) δ 11.17 (s, 1H), 9.55 (s, 1H), 7.35 (t, J = 2.8 Hz, 1H), 7.30 (d, J = 8.0 Hz, 1H), 7.05 (t, J = 7.7 Hz, 1H), 6.89 (d, J = 7.2 Hz, 1H), 6.61 (s, 1H), 3.94-3.81 (m, 1H), 3.14-3.00 (m, 2H), 2.7-2.73 (m, 2H); MS: 254.1 [M − H]. | 0.51 | 13.4 |

Data of the fluorescence polarization assays and protein thermal shift assays of some comparative compounds having a certain activity on inhibiting of LC3B are listed in Table 2. The comparative compounds were found during the study and have a certain activity on the inhibition of LC3B with an IC₅₀ value between 25 μM and 50 μM. The main difference between the comparative compounds and the compounds having a higher inhibitory activity against LC3B according to the present invention is that the comparative compounds do not contain the structure of “α,β-unsaturated carbonyl.”

TABLE 2
Data of the fluorescence polarization assays and protein thermal shift assays

| Comparative | | IC₅₀ | ΔT |
|---|---|---|---|
| Compounds | Structures | (μM) | (° C.) |
| 1 | 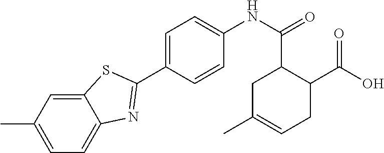 [SMILES: CC1=CCC(C(=O)O)C(C(=O)Nc2ccc(-c3nc4ccc(C)cc4s3)cc2)C1] | 46 | 0 |
| 2 | 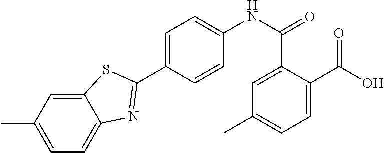 [SMILES: Cc1ccc(C(=O)O)c(C(=O)Nc2ccc(-c3nc4ccc(C)cc4s3)cc2)c1] | 50 | 0 |
| 3 | 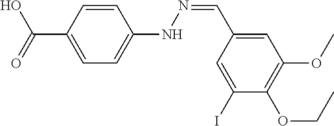 [SMILES: CCOc1c(I)cc(/C=N\Nc2ccc(C(=O)O)cc2)cc1OC] | 28 | 2.1 |
| 4 | 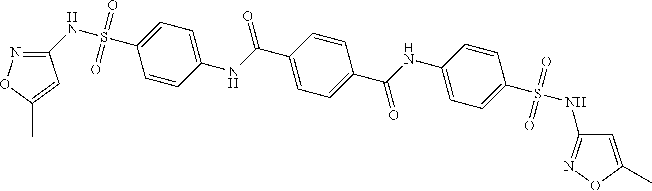 [SMILES: Cc1cc(NS(=O)(=O)c2ccc(NC(=O)c3ccc(C(=O)Nc4ccc(S(=O)(=O)Nc5cc(C)on5)cc4)cc3)cc2)no1] | 28 | 1.8 |
| 5 | 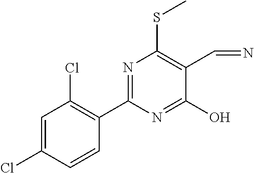 [SMILES: CSc1nc(-c2ccc(Cl)cc2Cl)nc(O)c1C=N] | 25 | 0 |
| 6 | 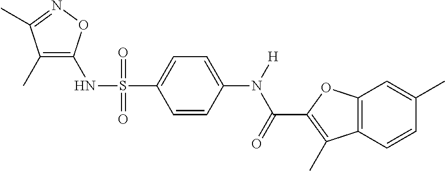 | 47 | 0 |

The above data of the fluorescence polarization assays indicates that the compound SM-LG of the present invention has a higher activity against LC3B when compared with the comparative compounds. The data of the protein thermal shift assays demonstrates that the compound SM-LG of the present invention is covalently bound with LC3B to form an LC3B covalent complex with a higher protein thermodynamic stability. While the protein thermodynamic stability is almost not increased after LC3B being treated with the comparative compounds, which shows that the comparative compound do not covalently bind with LC3B to form an LC3B covalent complex. As the main difference between the comparative compounds and the compounds having a higher inhibitory activity against LC3B according to the present invention is that the comparative compounds do not contain the structure of “α,β-unsaturated carbonyl”, the LC3B covalent complex formed by covalently binding the compound SM-LG of the present invention with LC3B has a specificity. And the structure of “α,β-unsaturated carbonyl” may probably play an important role during the procedure for forming the LC3B covalent complex by covalently binding the compound SM-LG of the present invention with LC3B.

### Crystallization and Resolution of Crystal Complex Structure of LC3B Protein and Small Molecule

For the purpose of demonstrating the compound SM-LG covalently binding with LC3B to form an LC3B covalent complex, Compound A and compound I was used for subsequent protein crystallization experiments and structural analysis.

A vacant protein crystal of LC3B (2-119) (SEQ ID NO: 4) was obtained by the sit-drop method, and then the crystal was taken out and soaked in a bath containing Compound A with a final concentration of 1-5 mM. The diffraction data were collected at the 19U1 line station of Shanghai Synchrotron Radiation Facility. And the diffraction data were integrated by XDS software and then compressed by Aimless module in CCP4. The LC3B protein structure with a PDB number of 3VTU was used as a template to perform molecular replacement by Phaser module to obtain the initial phase information, and then final refinement was performed by PHENIX and COOT.

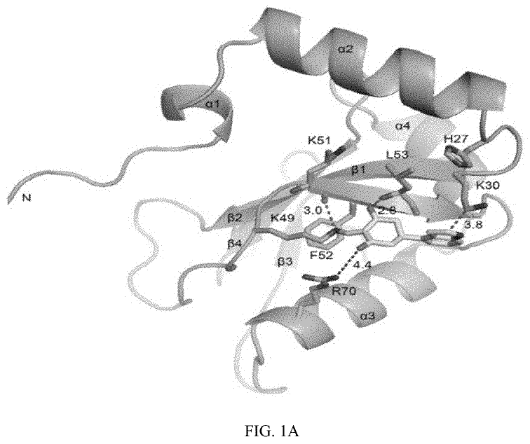

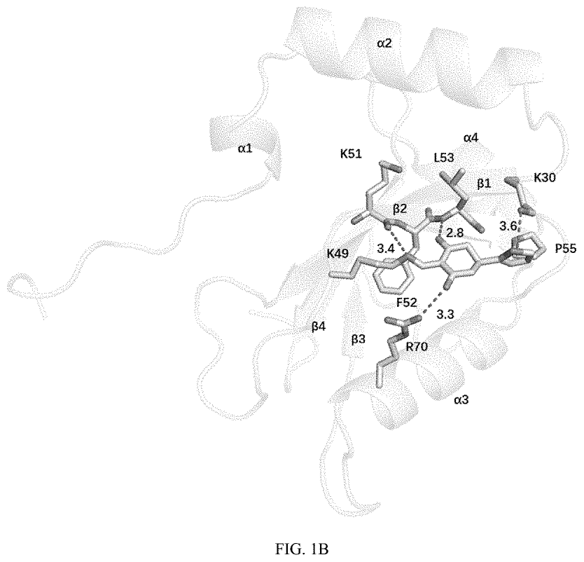

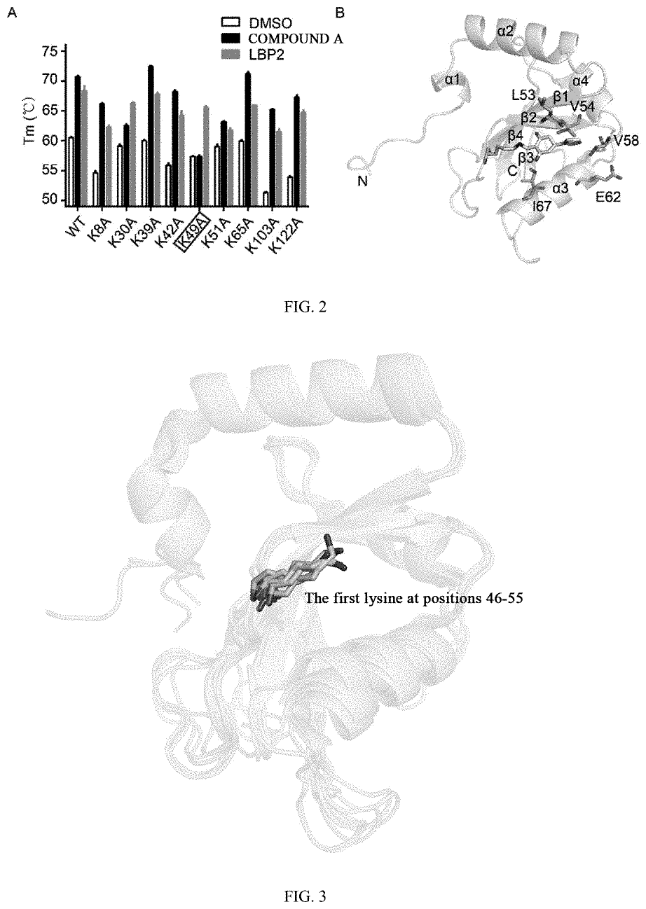

FIG. 1A shows a covalent complex of Compound A and LC3B; FIG. 1B shows a covalent complex of Compound I and LC3B. Compound A and compound I are covalently linked to the lysine residue (ε-amino group) at position 49 in LC3B. As shown in FIGS. 1A and 1B, the modified lysine residue at position 49 interacts with its surrounding amino acid residues (black dotted line; the unit of distance is angstrom).

Adjacent to the pocket L of LC3B, the lysine at position 52 and the arginine at position 70 provide a strong basic environment for obtaining a stable binding conformation of the cyclohexanedione moiety of Compound A and compound I. In this basic environment, the cyclohexanedione moiety is reacted with the lysine residue at position 49, and then the N-containing leaving moiety of Compound A is left to form a covalent complex of Compound A and LC3B as well as a covalent complex of Compound I and LC3B. The covalent complex has the following structure:

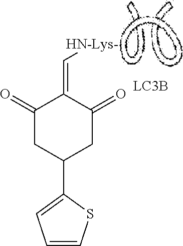

(using the covalent complex of Compound A and LC3B as an example). In this structure, HN-Lys- represents the ε-amino group of the lysine at position 49 in LC3B.

The covalent complex of Compound A and LC3B, the covalent complex of Compound I and LC3B, and other covalent complexes with similar structure can play a role in diagnosing and treating diseases associated with LC3B. For example, this covalent complex can be used as a biomarker for diagnosing and treating diseases associated with LC3B.

In the structure of the covalent complex of Compound A and LC3B, the presence of the cation-π interaction results in the thiophene ring moiety to be locked by the ionized lysine at position 30 (calculated by H++). In addition, the modified lysine at position 49 can respectively form a hydrogen bond with the leucine at position 53, the lysine at position 51 and the arginine at position 70 in LC3B, which are closely related to the good affinity and conformational stability of the compound. The covalent complex of Compound I and LC3B also has similar performance.

Similar to Compound A, Compounds B, C, D, E, F, and G can also be covalently linked to the lysine residue (ε-amino) at position 49 in LC3B to form covalent complexes, wherein the covalent complexes formed by Compounds B, C, D have the following structures:

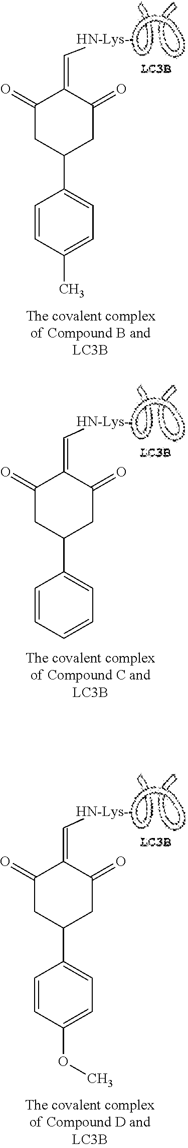

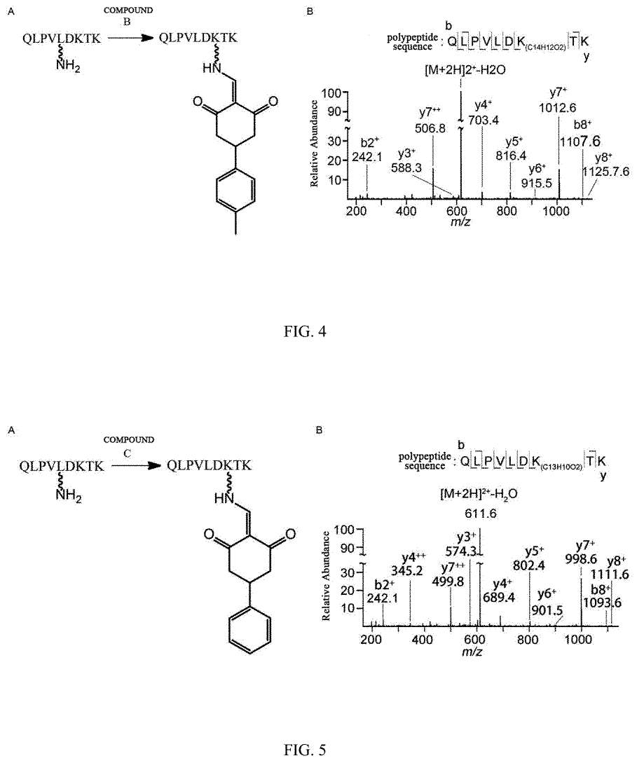

The above three covalent complexes are also verified by mass spectrometry data, and the analytical data of the mass spectrum is shown in FIGS. 4-6, which will be discussed in detail below.

The three covalent complexes can also play a role in diagnosing and treating diseases associated with LC3B. For example, this covalent complex can be used as a biomarker for diagnosing and treating diseases associated with LC3B.

Data from the protein thermal shift assay will also indicate that the thermodynamic stability of proteins of the above mammalian ATG8 homologue covalent complexes is different from the thermodynamic stability of proteins of LC3B. The melting temperature of the above covalent complex may be 2° C. higher than the melting temperature of LC3B. Preferably, the melting temperature of the above covalent complex may be 5° C. higher than the melting temperature of LC3B. The thermodynamic stability of the covalent complex can be used to detect the covalent complex and can be used to diagnose and treat diseases associated with LC3B.

### The Selectivity of Compound A for the Lysine at Position 49 in LC3B

LC3B totally has 9 lysines, and for investigating the site selectivity of Compound A, all the 9 lysines were mutated to alanine (K8A, K30A, K39A, K42A, K49A, K51A, K65A, K103A, K122A; SEQ ID NO:7-15). Protein thermal shift analysis for all mutants revealed that, except for the K49A mutant, Compound A has caused a significant thermal shift to all other mutants. The positive control polypeptide, LBP2 (SEQ ID NO: 6), has caused significant heat shift to all mutants.

The data of selectivity verified that Compound A can selectively modify the lysine at position 49 and would not modify other lysines in LC3B.

The lysine at position 49 in LC3B is present in all proteins of the mammalian ATG8 homologous family. FIG. 3 shows the first lysine at positions 46-55 is highly conservative in the proteins of the mammalian homologous family (LC3A, LC3B, LC3C, GABARAP, GABARAPL1 and GABARAPL2; respective PDB ID: 3WAL, 3VTU, 3WAM, 1GNU, 2R2Q and 4C07). Thus, the compound SM-LG can effectively and covalently modify the first lysine at positions 46-55 of the ATG8 mammalian homologous family protein.

The following documents are used as references for implementing the above embodiments.
Kabsch, W. XDS. Acta Cryst. D66, 125-132 (2010)
M. D. Winn et al. Overview of the CCP4 suite and current developments. Acta. Cryst. D67, 235-242 (2011)
Emsley, P. & Cowtan, K. Coot: model-building tools for molecular graphics. Acta Crystallogr. D 60, 2126-2132 (2004)
Adams, P. D. et al. PHENIX: building a new software for automated crystallographic structure determination. Acta Crystallogr. D 58, 1948-1954 (2002)

### Mass Spectrometry

For further verifying the covalent binding, LC3B (1-125) (SEQ ID NO: 3) protein was separately incubated with compounds B, C and D for mass spectrometry.

After incubating the compound and protein for a given period of time, the product was separated by gel electrophoresis, and the protein having an appropriate size was digested by trypsin. The resulting polypeptide was dissolved and loaded onto a C18 reverse-phase column coupled to an EASY-nLC 1000 system. The polypeptide was eluted to perform mass spectrometry and Mascot search.

As shown in FIG. 4, the mass spectrometry confirmed that Compound B covalently modified the lysine at position 49 in LC3B. (A) Reaction mechanism; (B) Ions of type b and type y. According to the mass analysis of y3 and b8, the modification is inferred to occur on the lysine at position 49, and the portion modified on compound B corresponds to the chemical composition of C₁₄H₁₂O₂.

As shown in FIG. 5, the mass spectrometry confirmed that Compound C covalently modified the lysine at position 49 in LC3B. (A) Reaction mechanism; (B) Ions of type b and type y. According to the mass analysis of y3 and b8, the modification is inferred to occur on the lysine at position 49, and the portion modified on compound C corresponds to the chemical composition of C₁₃H₁₀O₂.

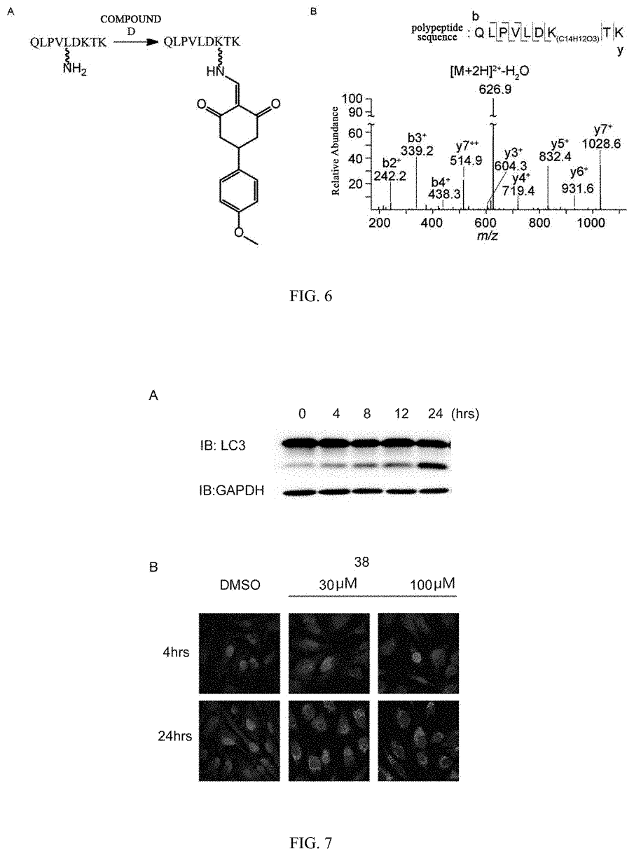

As shown in FIG. 6, the mass spectrometry confirmed that Compound D covalently modified the lysine at position 49 in LC3B. (A) Reaction mechanism; (B) Ions of type b and type y. According to the mass analysis of y3, the modification is inferred to occur, and the portion modified on compound D corresponds to the chemical composition of C₁₄H₁₂O₃.

As shown in FIGS. 4, 5 and 6, the mass spectrometry data verified the covalent modification of the LC3B protein by the compound SM-LG.

### Autophagy

For investigating the effect of compounds on autophagy, Hela cells were seeded into a 6-well plate, cultured overnight, treated with 30 μM or 100 μM of Compound B for 12 h, then replaced to a serum-free medium and starved for 24 hours. The medium was aspirated out, washed once with PBS, and the cells were lysed by a 2× loading buffer of SDS-PAGE. The samples were boiled at 99° C. for 10 minutes, separated by SDS-PAGE and then performed LC3-I/LC3-II assay by LC3B antibody (Novus).

As shown in FIG. 7A, LC3B accumulated as the treatment time of compound increases.

For further investigating the effects of compounds on cell autophagosomes, Hela cells were seeded onto glass coverslips in 6-well plate and cultured until the cells being in good conditions, which were treated with 30 μM or 100 μM of Compound B for 12 h, then replaced to a serum-free medium and starved for 24 hours. The cells were pre-cooled for 10 minutes, then punched with 0.2% Triton X-100 and stood at room temperature for 10 minutes. Next, the cells were blocked with PBS containing 2.5% BSA and incubated overnight with a 4 degree anti-LC3B primary antibody, after which the primary antibody was recognized with a fluorescent secondary antibody, the nuclei were stained with DAPI and photographed under a microscope. As shown in FIG. 7B, as compared with the control group, the cell autophagosomes accumulated after being treated with Compound 38, and the higher the concentration, the more the accumulation.

It should be understood that it will be apparent to those skilled in the art that various changes and modifications may be made by those skilled in the art without departing from the scope of the invention. All these equivalent modifications fall into the scope defined by the appended claims of the application.

1. A method for modulating a mammalian ATG8 homologue, comprising: 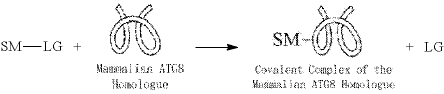
providing a compound SM-LG including a moiety SM- having a function of modulating a mammalian ATG8 homologue and a leaving moiety -LG;
the compound SM-LG reacts with a mammalian ATG8 homologue to produce a covalent complex of the mammalian ATG8 homologue.

2. The method according to claim 1, wherein the reaction of the compound SM-LG with a mammalian ATG8 homologue is a substitution reaction.

3. The method according to claim 1, wherein LG-H is a small molecule compound; and SM- has a structure of α,β-unsaturated carbonyl.

4. A covalent complex of a mammalian ATG8 homologue, having the following structure: 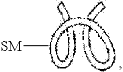
wherein, [symbol]
is a mammalian ATG8 homologue,
SM- is a moiety having a function of modulating a mammalian ATG8 homologue.

5. The method according to claim 1, wherein SM- is linked to the mammalian ATG8 homologue by a covalent bond.

6. The method according to claim 5, wherein SM- is linked to the ε-amino group of the first lysine at positions 46-55 in the mammalian ATG8 homologue by a covalent bond, as shown in the following formula: 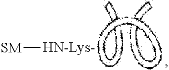
wherein HN-Lys- represents the ε-amino group of the first lysine at positions 46-55 in a mammalian ATG8 homologue.

7. The method according to claim 6, wherein the mammalian ATG8 homologue is LC3B, preferably SM is linked to the ε-amino group of the lysine at position 49 in LC3B by a covalent bond.

8. The method according to claim 1, wherein said SM- has the structure as shown in the following general formula Ia: 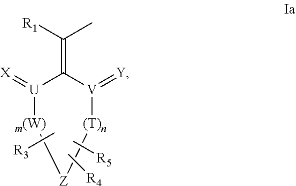
in the general formula Ia:
X and Y are each independently selected from the group consisting of O, S, NRₐ, NOH, and CH₂;
U and V are each independently selected from the group consisting of C, S, SO, and PORₐ;
W, Z, and T are each independently selected from the group consisting of O, S, SO, SO₂, N, NRₐ, CO, C, CRₐ, and CH₂;
Rₐ is H or C1-6 alkyl;
m is 0, 1, 2, or 3;
n is 0, 1, 2, or 3;
R₁ is selected from the group consisting of H, deuterium, unsubstituted C1-6 alkyl or C1-6 alkyl substituted by a substituent selected from hydroxyl and halogen, unsubstituted phenyl or phenyl substituted by a substituent selected from halogen, hydroxyl, C1-C6 alkyl and C1-C6 heteroalkyl;
R₃, R₄, and R₅ are each independently selected from the group consisting of H; hydroxyl; amino group; halogen; cyano; nitro; carboxyl; formyl; amide group; ester group; unsubstituted C1-6 alkyl or C1-6 alkyl substituted by a substituent selected from hydroxyl, halogen and C1-6 alkoxy; C1-6 heteroalkyl; C2-6 alkenyl; C2-6 alkynyl; substituted or unsubstituted —CONH₂—(C6-10 aryl); substituted or unsubstituted —CH═CH—(C6-10 aryl); substituted or unsubstituted C6-10 aryl; substituted or unsubstituted 5-10 membered heteroaryl; substituted or unsubstituted C3-10 cycloalkyl; substituted or unsubstituted C3-10 cycloalkenyl; substituted or unsubstituted 3-10 membered heterocycloalkyl; substituted or unsubstituted 3-7 membered heterocycloalkenyl; substituted or unsubstituted C6-10 aryl C1-6 alkyl; substituted or unsubstituted C1-6 alkyl C6-10 aryl; substituted or unsubstituted 5-10 membered heteroaryl C1-6 alkyl; and substituted or unsubstituted C1-6 alkyl 5-10 membered heteroaryl;
or two adjacent groups of R₃, R₄ and R₅ may be bonded to form a substituted or unsubstituted C6-10 aryl group, a substituted or unsubstituted 5-10 membered heteroaryl group, a substituted or unsubstituted C3-10 cycloalkyl group, or a substituted or unsubstituted 3-10 membered heterocycloalkyl group;
the “substituted” in “substituted or unsubstituted” means that being substituted by one or more substituents selected from the group consisting of H, hydroxyl, amino group, cyano, nitro, carboxyl, halogen, C1-6 alkyl, C1-6 haloalkyl or C1-6 hydroxyalkyl;
and meets one of the following conditions:
(1) when W, Z or T is substituted by one group of R₃, R₄ and R₅, the W, Z or T is N or CH;
(2) when W, Z or T is substituted by one group of R₃, R₄ and R₅ and this group is bonded to another adjacent group of R₃, R₄ and R₅ to form a substituted or unsubstituted C6-10 aryl or a substituted or unsubstituted 5-10 membered heteroaryl, the W, Z or T is C;
(3) when W, Z or T is substituted by two of R₃, R₄ and R₅, the W, Z or T is C.

9. The method according to claim 8, wherein the general formula Ia is the following general formula IIa: 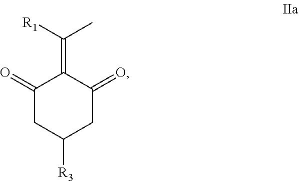
wherein, R₁ is selected from the group consisting of H, deuterium, unsubstituted C1-6 alkyl or C1-6 alkyl substituted by a substituent selected from hydroxyl and halogen, and unsubstituted phenyl or phenyl substituted by a substituent selected from halogen, hydroxyl, C1-C6 alkyl and C1-C6 heteroalkyl;
R₃ is selected from the group consisting of H; hydroxyl; amino group; halogen; cyano; nitro; carboxyl; formyl; amide group; ester group; unsubstituted C1-6 alkyl or C1-6 alkyl substituted by a substituent selected from hydroxyl, halogen and C1-6 alkoxy; C1-6 heteroalkyl; C2-6 alkenyl; C2-6 alkynyl; substituted or unsubstituted —CONH₂—(C6-10 aryl); substituted or unsubstituted —CH═CH—(C6-10 aryl); substituted or unsubstituted C6-10 aryl; substituted or unsubstituted 5-10 membered heteroaryl; substituted or unsubstituted C3-10 cycloalkyl; substituted or unsubstituted C3-10 cycloalkenyl; substituted or unsubstituted 3-10 membered heterocycloalkyl; substituted or unsubstituted 3-7 membered heterocycloalkenyl; substituted or unsubstituted C6-10 aryl C1-6 alkyl; substituted or unsubstituted C1-6 alkyl C6-10 aryl; substituted or unsubstituted 5-10 membered heteroaryl C1-6 alkyl; and substituted or unsubstituted C1-6 alkyl 5-10 membered heteroaryl;
the “substituted” in “substituted or unsubstituted” means that being substituted by one or more substituents selected from the group consisting of H, hydroxyl, amino group, cyano, nitro, carboxyl, halogen, C1-6 alkyl, C1-6 haloalkyl or C1-6 hydroxyalkyl.

10. The method according to claim 9, wherein,
R₃ is selected from the following groups: 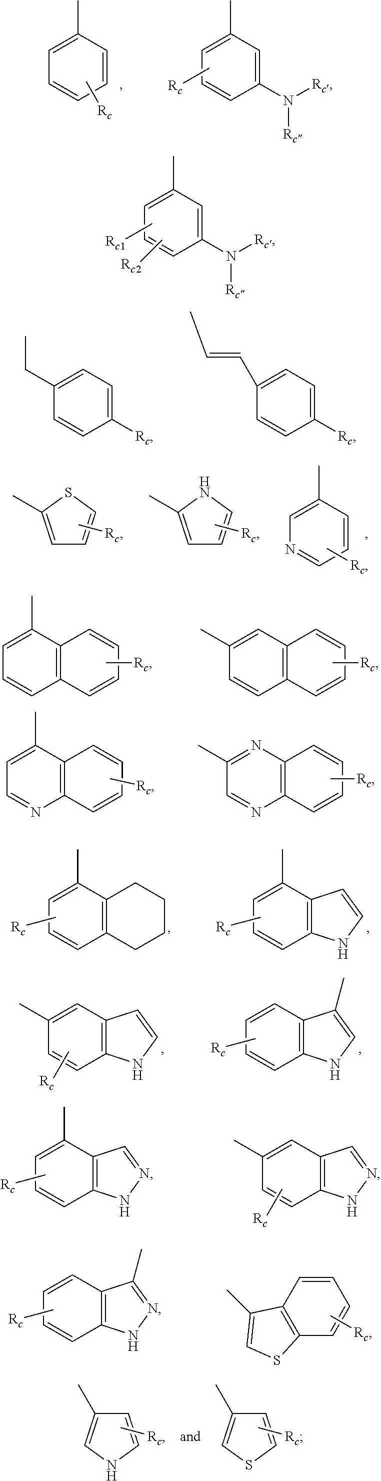
wherein,
R_{c}, R_{c1}, R_{c2}, Rc′ and Rc″ are each independently selected from the group consisting of H, hydroxyl, amino group, NRaRa′, halogen, cyano, nitro, carboxyl, formyl, amide group, ester group, C1-6 haloalkyl, C1-6 hydroxyalkyl, C1-6 heteroalkyl, C1-6 alkoxy, C1-6 alkoxyalkyl, C2-6 alkenyl, C2-6 alkynyl, C6-10 aryl, 5-10 membered heteroaryl, C3-10 cycloalkyl, 3-10 membered heterocycloalkyl, 3-7 membered heterocycloalkenyl, C1-6 alkyl C6-10 aryl, 5-10 membered heteroaryl C1-6 alkyl or C1-6 alkyl 5-10 membered heteroaryl; preferably selected from the group consisting of H, hydroxyl, amino group, NRaRa′, halogen, carboxyl, formyl, amide group, ester group, C1-6 haloalkyl, C1-6 hydroxyalkyl, C1-6 heteroalkyl, C1-6 alkoxyl, C3-10 cycloalkyl, 3-10 membered heterocycloalkyl, substituted or unsubstituted phenyl or pyridyl;
Rₐ is H or C1-6 alkyl;
or R_{c1} and R_{c2} may be bonded to form C6-10 aryl, 5-10 membered heteroaryl, C3-10 cycloalkyl, or 3-10 membered heterocycloalkyl;
or R₃ is selected from the following groups: 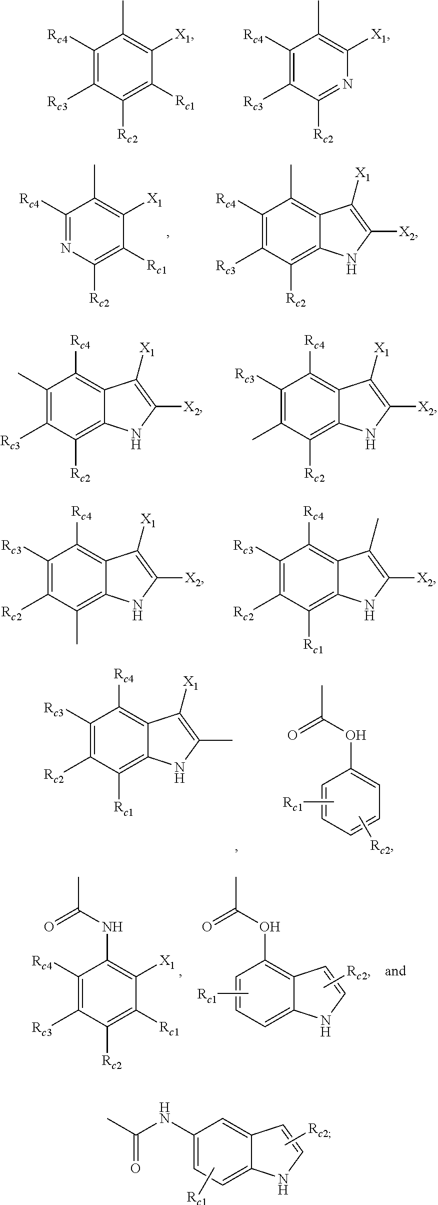
wherein, X₁ is F, Cl, Br, I or trifluoromethyl;
X₂ is H, F, Cl, Br, or I;
R_{c1}, R_{c2}, R_{c3} and R_{c4} are each independently selected from the group consisting of H, hydroxyl, amino group, NRaRa′, halogen, cyano, nitro, carboxyl, formyl, amide group, ester group, C1-6 haloalkyl, C1-6 hydroxyalkyl, C1-6 heteroalkyl, C1-6 alkoxy, C1-6 alkoxyalkyl, C2-6 alkenyl, C2-6 alkynyl, C6-10 aryl, 5-10 membered heteroaryl, C3-10 cycloalkyl, 3-10 membered heterocycloalkyl, 3-7 membered heterocycloalkenyl, C1-6 alkyl C6-10 aryl, 5-10 membered heteroaryl C1-6 alkyl and C1-6 alkyl 5-10 membered heteroaryl; preferably selected from the group consisting of H, hydroxyl, amino group, NRaRa′, halogen, carboxyl, formyl, amide group, ester group, C1-6 haloalkyl, C1-6 hydroxyalkyl, C1-6 heteroalkyl, C1-6 alkoxyl, C3-10 cycloalkyl, 3-10 membered heterocycloalkyl, substituted or unsubstituted phenyl or pyridyl;
Rₐ is H or C1-6 alkyl;
or R_{c1} and R_{c2}, or R_{c2} and R_{c3}, or R_{c3} and R_{c4} may be bonded to form C6-10 aryl, 5-10 membered heteroaryl, C3-10 cycloalkyl, and 3-10 membered heterocycloalkyl.

11. The method according to claim 1, wherein the covalent complex of the mammalian ATG8 homologue has a melting temperature that is at least 2° C. higher than the mammalian ATG8 homologue.

12. (canceled)

13. (canceled)

14. The method according to claim 2, wherein the reaction of the compound SM-LG with a mammalian ATG8 homologue is a nucleophilic substitution reaction.

15. The method according to claim 3, wherein LG-H is a water molecule.

16. The covalent complex of a mammalian ATG8 homologue according to claim 4, wherein SM- is a moiety having a structure of α,β-unsaturated carbonyl.

17. The method according to claim 11, wherein the covalent complex of the mammalian ATG8 homologue has a melting temperature that is at least 5° C. higher than the mammalian ATG8 homologue.
